(19) United States
(12) Patent Application Publication
(10) Pub. No.: US 2010/0286097 A1
(43) Pub. Date: Nov. 11, 2010

(54) INSECTICIDAL COMPOSITIONS

(75) Inventors: Sharon Rose Mercieca, Baxter (AU); Kerrie-Anne Lee Mercieca, Baxter (AU)

Correspondence Address: CHERNOFF, VILHAUER, MCCLUNG & STENZEL, LLP, 601 SW Second Avenue, Suite 1600, PORTLAND, OR, 97204-3157, US

(73) Assignee: S.R.M. PTY. LTD., Baxter (AU)

(21) Appl. No.: 12/839,299

(22) Filed: Jul. 19, 2010

Related U.S. Application Data
(62) Division of application No. 11/575,053, filed on Apr. 5, 2007.

(30) Foreign Application Priority Data
Sep. 10, 2004 (AU) 2004905230
Mar. 16, 2005 (AU) 2005901290

(51) Int. Cl. A61K 31/60; A61P 33/14

(52) U.S. Cl. 514/159

(57) ABSTRACT

This invention relates to topical compositions for treating ectoparasites such as head lice and fleas. The composition comprises an ovicidally effective quantity of salicylic acid (typically 1-10% by weight) dispersed in a liquid vehicle or carrier. The vehicle may be an oily carrier including a mineral oil such as paraffin oil or a vegetable oil such as olive oil or castor oil or an alcoholic sulphate free shampoo base.

### FIELD OF THE INVENTION

This invention concerns preparations for treating infestations of head lice and fleas.

### BACKGROUND OF THE INVENTION

Such infestations are most common in children of school age. The insects move from person to person in close confines such as classrooms and play areas. The mature insects lay eggs which hatch in the hair and behind the ears and therefore renew the infestation continuously unless treated. Hair is an attractive habitat for the insects. Once the sebum of the scalp spreads into the hair, the eggs in particular become difficult to dislodge.

Head lice preparations commonly contain insecticides. Compositions for removing or loosening the eggs of head lice may contain enzymes for acting upon the cement extruded by the eggs to keep the eggs adhered to the sites where they were laid. These water-based enzyme compositions are not popular because sufferers suspect that enzymes will cause deterioration of the hair. U.S. Pat. No. 5,557,665 discloses such compositions.

U.S. Pat. No. 3,023,144 describes compositions for application to the human and animal skin for controlling fungal and bacterial infections. The chief constituent of these is citrus oils of which the active constituents are a-Limonene and p-cymene. The compositions contain olive oil and 0.25%-10% by weight of salicylic acid stating that the latter is included for its germicidal action and the skin sloughing effect of salicylic acid is diminished by the citrus oil.

U.S. Pat. No. 6,524,604 describes a composition for removal of lice ova from the hair of human subjects by applying gels made of xanthan gum, silicone conditioners and a small quantity of an organic acid such as acetic acid or citric acid. The patent describes the use of 3% salicylic acid as a pH adjuster. The main constituent being dimethyl isosorbide (DMI). These compositions decreased the force required to remove nits from human hair using a fine tooth comb.

### SUMMARY OF THE INVENTION

The usual composition aspect of this invention provides a topical composition for treating ectoparasitic insect infestation comprising an ovicidally effective quantity of salicylic acid dispersed in a liquid vehicle.

The quantity of salicylic acid may be 1-10%, preferably 5-8%. Compositions containing 5, 6, 7 or 8% by weight of the volume of the compositions are ovicidally effective. The compositions may be in any physical form suitable for application to the scalp and hair. Lotions, gels and creams are all suitable. The compositions may in addition include an emulsifier, a preservative/stabiliser, a surfactant, a fragrance and a colouring agent. The vehicle may be an oily carrier including a mineral oil such as paraffin oil or a vegetable oil such as olive oil or caster oil. Oil wets the eggs and remains in contact until washed out.

Oil based and water based compositions are less effective at concentrations below 4 or 5% and lower kills result. Compositions with higher concentrations of salicylic acid than 8% produce effective kills but may produce skin sloughing. As the subjects for lice treatment are mostly younger children, this is a skin effect best avoided.

The shampoo base may be an alcoholic soap solution. Solutions useful for this purpose may contain substantially equal quantities of ethyl or methyl alcohol and soft soap. The base is a mobile gel.

As soft soap shampoos tend to dry the skin, a small quantity of vegetable oil (3-8%) may be added. This facilitates combing of hair after shampooing human hair. The inclusion of oil in washes for animals is not so important.

The method of application is as follows. The gel is poured onto the hair of the scalp and rubbed into the hair with the fingers. The gel is left in the hair for 1-2 hours. Movement by the lice is seen initially but this subsides after 10-15 minutes and after 20 minutes all the lice appear motionless.

After 2 hours from the application of the gel, the hair is rinsed in water to remove eggs and dead lice. The hair is towel dried and left to dry fully. The treatment is repeated after 7 days to meet the possibility that live eggs have remained after the first treatment.

It is not certain what role the alcoholic component of the soap solution takes. It is assumed that the salicylic acid kills the lice as they suspend respiration during the immersion reflex by which lice survive immersion in liquid. The lice are thus prevented from taking a blood meal after immersion. The juveniles particularly need to feed early after immersion and it is thought that they are especially vulnerable to the combination of shampoo and salicylic acid.

### Shampoo

| | |
|---|---|
| Salicylic acid | 6 g |
| 95% alc | 50 ml |
| Base (SO₄″ free) | 50 ml |

### EXAMPLE

### Sulphate Free Shampoo

| | | |
|---|---|---|
| Salicylic acid | 6 | g |
| 95% Ethyl alcohol | 50 | ml |
| Melrose ‘Everyday’ sulphate-free | to 100 | ml |
| shampoo base | 100 | ml |

### Procedure

The salicylic acid is dissolved in the alcohol, then added to the shampoo base and mixed. Sodium lauryl sulphate which would otherwise be a suitable base is avoided because it precipitates upon addition of the alcoholic solution of salicylic acid.

### EXAMPLE

### Alcoholic Soap Solution

| | | |
|---|---|---|
| Salicylic acid | 6 | g |
| Castor oil (5 g) | 5 | ml |
| Ethyl Alchol 95% (309) | 30 | ml |
| Alcoholic soap solution | to 100 | ml |
| | 100 | ml |

### Procedure

The 95% alcohol is mixed with most of the alcoholic soap solution which dissolves the salicylic acid with stirring. When dissolved, the castor oil is added plus the remainder of the soap solution.

This preparation was used as a dog wash. The animal was confined in a mechanical dog wash. The solution was rubbed into the fur for several minutes while the operator talked to the dog and soothed it. The solution was then rinsed off with water pumped through a rose. The dog was dried with a towel but remained wet for a further 40 minutes at ambient temperature (21° C.).

### EXAMPLE

### Castor/Olive Oil Version

This is an oily preparation of salicylic acid in castor/olive oil.

| | | |
|---|---|---|
| Salicylic acid | 12 | g |
| Butylated hydroxytoluene (0.1%) | 0.2 | g |
| Vitamin E oil 0.1% | 0.2 | ml |
| Equivalent to 50 IU D-1-a-tocopherol acetate | | |
| Olive oil (3.0%) | 60 | ml |
| Castor oil to (≈70%) | 200 | ml |

### Procedure

A 70:30 mix of castor/olive oil is warmed to 50° C. The salicylic acid, BHT and Vitamin E oil are dissolved with stirring into the oil mix.

Testing the effectiveness of the treatment solution was carried out as follows:

From a population of body lice adults, nymphs and eggs, the tester randomly selected males and females. 100 vigorous adults were inserted into a syringe wherein the needle exit was blocked with gauze. The lice tend to stay in contact with the gauze. The sample treatment solution was added in an amount to wet them all and they were left at ambient temperature (32° C.) for 10 minutes. After the contact with the solution they were released from the syringe and retained on the gauze and washed with water for 60 seconds to remove the solution. They were dried gently as a group with tissue and deposited in a shallow dish again on a cloth surface (about 50 to a dish). The dishes were placed in an incubator and after one hour they were inspected for activity. They were returned to the incubator and recovered 17 hours later and classified as moribund level one or moribund level two.

### Lice Classification

Alive: able to move and right itself when rolled on back.

Moribund (Level 1): when rolled on back is unable to right itself.

Moribund (Level 2): when rolled on back is unable to right itself. Can only just move legs and antennae when prodded.

Dead: no movement.

Lice which are moribund after 17 hours are unlikely to survive in the hair of a subject.

| | 1 hr | 17 hr |
|---|---|---|
| Adults & 3^{rd} stage nymphs used, time checks | | |
| Corrected Mortality | 85.4 | 87.8 |

Corrected Mortality = ((% alive in control- % alive in test)/%
alive in control)*100

| Date | Jan. 06, 2005 | | |
|---|---|---|---|
| substance | Acid 6% in olive oil and Castor oil | | |
| | Preservatives; BHT 0.1% Vit E. | | |
| | Batch: 180305/8 | | |
| Concentration | as supplied | | |
| treatment | dipping 10 min 32° C. in sample; rinse | | |
| | 1 min in 32° C. water | | |
| Time | | | |
| Replicate 1 | Alive | 7 | 6 |
| 50 lice | % | 14 | 12 |
| | Moribund_level 1 | 0 | 1 |
| | % | 0 | 2 |
| | Moribund_level 2 | 4 | 1 |
| | % | 8 | 2 |
| | Dead | 39 | 42 |
| | % | 78 | 84 |
| Replicate 2 | Alive | 5 | 4 |
| 50 lice | % | 10 | 8 |
| | Moribund_level 1 | 0 | 0 |
| | % | 0 | 0 |
| | Moribund_level 2 | 5 | 1 |
| | % | 10 | 2 |
| | Dead | 40 | 45 |
| | % | 80 | 90 |
| Replicate 3 | Alive | 9 | 7 |
| 50 lice | % | 18 | 14 |
| | Moribund_level 1 | 0 | 0 |
| | % | 0 | 0 |
| | Moribund_level 2 | 4 | 0 |
| | % | 8 | 0 |
| | Dead | 37 | 43 |
| | % | 74 | 86 |
| Replicate 4 | Alive | 8 | 7 |
| 50 lice | % | 16 | 14 |
| | Moribund_level 1 | 0 | 0 |
| | % | 0 | 0 |
| | Moribund_level 2 | 4 | 0 |
| | % | 8 | 0 |
| | Dead | 38 | 43 |
| | % | 76 | 86 |
| Total | Alive | 29 | 24 |
| 200 lice | % | 14.5 | 12 |
| | Moribund_level 1 | 0 | 1 |
| | % | 0 | 0.5 |
| | Moribund_level 2 | 17 | 2 |
| | % | 8.5 | 1 |
| | Dead | 154 | 173 |
| | % | 77 | 86.5 |
| | % Dead + Moribund | 85.5 | 88.5 |

The ovicidal property of the treatment solutions was measured as follows:

Female body lice were given good conditions in which to lay eggs for 24-48 hours. The female lice were then separated from the eggs and the eggs inspected. Only healthy eggs were selected for the test. Those which were not damaged or squashed were selected and placed on gauze. The eggs were transferred into a micro plate assay container and the eggs were covered with treatment solution at ambient temperature. After 10 minutes, one batch was washed with water for 60 seconds and dried gently with tissue. The second batch was similarly treated after 20 minutes. The batches were transferred to dishes and kept in an oven at 32° C. for 9 days. Those which did not hatch were presumed dead.

Substance: Treated with Acid 6% in Olive oil & Castor oil
Preservatives: BHT0.1% VitE
Batch: 180305/8, rinsed with water for 1 min,
then swabbed with tissue

| Age of eggs (Hrs) | 24-48 (hrs) | | | | | |
|---|---|---|---|---|---|---|
| Dipping time (min) | 10 mins | | | | | |
| Temperature (° C.) | 31, incubator | | | | | |
| Humidity (%) | 50 | | | | | |
| Replicates | 1 | 2 | 3 | 4 | 5 | Total |

Cumulative eggs hatched Day 6

| | | | | | | |
|---|---|---|---|---|---|---|
| % | 0 | 0 | 0 | 0 | 0 | 0 |

Cumulative eggs hatched Day 7

| | | | | | | |
|---|---|---|---|---|---|---|
| % | 0 | 0 | 0 | 0 | 0 | 0 |

Cumulative eggs hatched Day 8

| | | | | | | |
|---|---|---|---|---|---|---|
| % | 0 | 0 | 0 | 0 | 0 | 0 |

Cumulative eggs hatched Day 9

| | | | | | | |
|---|---|---|---|---|---|---|
| % | 0 | 0 | 0 | 0 | 0 | 0 |

Cumulative eggs hatched Day 10

| | | | | | | |
|---|---|---|---|---|---|---|
| % | 0 | 1 | 2 | 3 | 1 | 7 |
| No. of eggs | 20 | 18 | 23 | 19 | 20 | 100 |
| % | 0.00 | 5.56 | 8.70 | 15.79 | 5.00 | 7.00 |

| | |
|---|---|
| Comments | Each replicate has 20 eggs if not specifically noted. |

Substance: Treated eggs with water, rinsed with
water for 1 min then swabbed with tissue

| | CONTROL | | | | | |
|---|---|---|---|---|---|---|
| Age of eggs (Hrs) | 24-48 (hrs) | | | | | |
| Dipping time (min) | 10 mins | | | | | |
| Temperature (° C.) | 31, incubator | | | | | |
| Humidity (%) | 50 | | | | | |
| Replicates | 1 | 2 | 3 | 4 | 5 | Total |

Cumulative eggs hatched Day 6

| | | | | | | |
|---|---|---|---|---|---|---|
| % | 0 | 0 | 0 | 0 | 0 | 0 |

Cumulative eggs hatched Day 7

| | | | | | | |
|---|---|---|---|---|---|---|
| % | 0 | 0 | 0 | 0 | 0 | 0 |

Cumulative eggs hatched Day 8

| | | | | | | |
|---|---|---|---|---|---|---|
| % | 0 | 0 | 0 | 0 | 0 | 0 |

Cumulative eggs hatched Day 9

| | | | | | | |
|---|---|---|---|---|---|---|
| % | 0 | 0 | 0 | 0 | 0 | 0 |

Cumulative eggs hatched Day 10

| | | | | | | |
|---|---|---|---|---|---|---|
| % | 16 | 15 | 18 | 16 | 16 | 81 |
| No. of eggs | 19 | 16 | 20 | 19 | 17 | 91 |
| % | 84.21 | 93.75 | 90.00 | 84.21 | 94.12 | 89.01 |

| | |
|---|---|
| Comments | Each replicate has 20 eggs if not specifically noted. |

These tests were conducted on body lice but the result is not expected to be different for head lice.

### Application to Human Subject

The lotion is poured on to the dry hair of a 2 year old child showing moderate infestation with head lice and eggs. Sufficient lotion is applied to cover the scalp and hair thoroughly. The quantity used depends on the length of the hair. The lotion is brushed to spread the same and the hair assumes a glossy appearance as the oily base spreads into the hair. The scalp is massaged for 5 minutes. At this stage the lice are observed trying to escape from the treated area.

The lotion is left on the scalp and hair for 2-8 hours, whereafter the eggs are removed with a fine tooth comb. The oily base darkens the eggs making them easier to see and to dislodge. The lotion is washed out with shampoo. Inspection follows daily and the treatment is repeated after 7-10 days to kill any live lice which have hatched from any remaining eggs not killed by the first treatment. The hair is inspected at 7 day intervals until the infestation is over. All members of the child's family were inspected and treated where necessary at the same time.

The above tests were repeated with the alcoholic soap solution. Egg kills of 67% were recorded with 10 minute contact. This would be normally regarded as insufficient but as the contact time would be 2-8 hours, a much greater kill would result.

The above tests were also repeated with the sulphate free shampoo. A good ovicidal result was obtained with no egg hatches recorded on day 11.

It is to be understood that the word “comprising” as used throughout the specification is to be interpreted in its inclusive form, ie. use of the word “comprising” does not exclude the addition of other elements.

It is to be understood that various modifications of and/or additions to the invention can be made without departing from the basic nature of the invention. These modifications and/or additions are therefore considered to fall within the scope of the invention.

1-10. (canceled)

11. A method of treating ectoparasitic insect infestation, comprising topically applying a composition that comprises an ovicidally effective quantity of salicylic acid dispersed in a liquid vehicle.

12. A method according to claim 11, wherein the quantity of salicylic acid is 1-10% by weight of the total composition.

13. A method according to claim 11, wherein the quantity of salicylic acid is 5-8% by weight of the total composition.

14. A method according to claim 11, wherein the vehicle is a vegetable oil.

15. A method according to claim 14, wherein the oil is castor oil.

16. A method according to claim 14, wherein the oil is olive oil.

17. A method according to claim 11, wherein the oil is a mineral oil.

18. A method according to claim 11, wherein the oil is paraffin oil.

19. A method according to claim 11, wherein the vehicle is an alcoholic sulphate free shampoo base.

20. A method of treating ectoparasitic insect infestation in a subject's hair, comprising topically applying a composition that comprises an ovicidally effective quantity of salicylic acid dispersed in a liquid vehicle to the subject's scalp, allowing the composition to remain in contact with the subject's hair for at least one hour, and subsequently rinsing the subject's hair.
